(19) United States
(12) Patent Application Publication
(10) Pub. No.: US 2015/0272931 A1
(43) Pub. Date: Oct. 1, 2015

(54) New Treatment

(71) Applicants: Jacques BOUVIER, (US); Steve NANCHEN, (US); Jean-Luc PERRET, (US)

(72) Inventors: Jacques Bouvier, St. Aubin (CH); Steve Nanchen, Basel (CH); Jean-Luc Perret, St. Aubin (CH)

(21) Appl. No.: 14/432,763

(22) PCT Filed: Oct. 24, 2013
(86) PCT No.: PCT/EP2013/072248

§ 371 Date: Apr. 1, 2015

(30) Foreign Application Priority Data
Oct. 26, 2012 (EP) 12190171.4

(51) Int. Cl. A61K 31/426; A23K 1/18; A23K 1/16; A61K 9/16

(52) U.S. Cl. CPC A61K 31/426; A61K 9/1676; A23K 1/188; A23K 1/1646

(57) ABSTRACT

The present invention concerns the use of clothianidin for controlling sea lice in a fish population, which comprises feeding clothianid to the fish population according to a specific regime as outlined in the specification and claims. The process is especially suited for the treatment of salmon and provides prolonged effective protection against sea lice at sea.

The present invention relates to the control of sea lice, for example Lepeophtheirus salmonis, Caligus elongatus and Caligus rogercresseyi, infestations in fish farming, which includes the application of the neonicotinoid clothianidin to the fish.

The basis of sea lice control in commercial salmonid farming is largely still a treatment with chemicals such as organophosphates, synthetic pyrethroids, chitin synthesis inhibitors, hydrogen peroxide or macrocyclic lactones such as emamectin benzoate. Developing resistance by sea lice against said commercial products presents a big threat to the fish industry; on the one hand higher doses of the compounds might be employed which accelerates the issue of resistance development and moreover has the potential to create environmental toxicology issues. On the other hand there is a desperate search for new chemicals and treatment schedules thereof.

EP0590425 discloses very broadly a method of combatting fish parasites by administering an agonist or antagonist of the nicotinergic acetylcholine receptors to the fish. The document discloses a number of those agonists or antagonists specifically, but is silent about clothianidin. In addition, no details about the application of said class of compounds, such as the amount being necessary to control fish parasites, are available, and no in vivo data are disclosed. The only working example concerns the in vitro activity of imidacloprid against sea lice in a sea water bath containing 1 ppm or 100 ppm of the active ingredient.

Clothianidin is a known insecticide which is used in plant protection and in the treatment of seeds. However, no application in the veterinary field has been reported until now.

Surprisingly, it has now been found that clothianidin is extremely efficient in eliminating sea lice infestations while being tolerated very well by the fish and being environmentally safe.

The present invention therefore provides a method for reducing or eliminating sea lice in a fish population, which comprises treating the fish with clothianidin or a veterinary acceptable salt thereof at a concentration of from 3 to 300 mg clothianidin per kg of fish biomass.

### DETAILED DESCRIPTION

Clothianidin, (E)-1-(2-Chlor-1,3-thiazol-5-ylmethyl)-3-methyl-2-nitroguanidin, CAS No. 210880-92-5 (formerly 205510-53-8), has the chemical formula

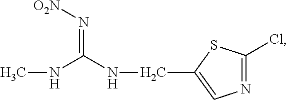

and may be applied in free form or in form of a veterinary acceptable salt. The above-given formula shows the molecule with trans-configuration of the nitro group relative to the thiazolylmethylamino moiety. However, the term clothianidin is also meant to encompass products which are mixtures of trans- and cis-isomer. It is preferred to apply a commercially available clothianidin product that either consists of the pure trans-isomer or is a mixture of trans-isomer with a small portion of cis isomer, for example 0% (w/w) cis isomer.

In accordance with this invention clothianidin is excellently suited for use in the control of fish-parasitic crustaceans. These include the Family Caligidae with representative genus Dissonus, Caligus (i.e. C. curtus, C. elongatus, C. clemensi, C. rogercresseyii), and Lepeophtheirus (i.e. L. salmonis); Families Cecropidae, Dichelesthiidae, Lernaeopodidae with representative genus Salmincola; Families Pandaridae, Pennellidae with representative genus Lernaeocera and Pennella; and Family Sphyriidae; Family Lernaeidae with representative genus Lernaea; Families Bomolochidae, Chondracanthidae, Ergasilidae and Philichthyidae; Family Argulidae with representative genus Argulus (i.e. A. foliaceus).

The fish include food fish, breeding fish, aquarium, pond, river, reservoir fish of all ages occurring in freshwater, sea water and brackish water. For example, bass, bream, carp, catfish, char, chub, cichlid, cod, eel, flounder, gourami, grayling, grouper, halibut, mullet, plaice, pompano, roach, rudd, salmon, sole, tilapia, trout, tuna, whitefish, yellowtail.

Clothianidin is particularly suitable for treating salmon. The term “salmon” within the scope of this invention will be understood as comprising all representatives of the family Salmonidae, especially of the subfamily salmoninae, and preferably, the Atlantic salmon (Salmo salar), rainbow trout (Oncorhynchus mykiss), brown or sea trout (S. trutta), the Pacific salmon, Cherry salmon or seema (O. masou), Taiwanese salmon (O. masou formosanum), chinook salmon or King salmon (O. tshawytscha), chum salmon or Calico salmon (O. keta), coho salmon or silver salmon (O. kisutch), pink salmon (O. gorbuscha), Sockeye salmon or Red salmon (O. nerka), artificially propagated species, such as Salmo clarkii, and Salvelinus species such as Brook trout (S. fontinalis).

Preferred objects of the present invention are the Atlantic and Pacific salmon and the sea trout including trout species, which are farmed at sea but not traditionally called “sea trout”.

The fish may be treated orally, e.g. via their feed. Moreover, the active ingredient may be applied by bath treatment, for example in a “medicinal bath” wherein the fish are placed and where they are kept for a period of time (minutes to several hours), for example, when being transferred from one breeding basin to another. It is also possible to treat the biotope of the fish temporarily or continuously, e.g the net cages, entire ponds, aquaria, tanks or basins in which the fish are kept. According to still a further embodiment, the fish are treated parenterally, for example by injection.

The active substance is administered in formulations which are adjusted to the specific application. A formulation for oral administration is, for example, a powder, granulate, solution, emulsifiable concentrate or suspension concentrate, in particular a medicated fish feed as described below. Formulations for bath application or for treating the biotope are powders, granulates, solutions, emulsions or suspensions, tablets or the active substance itself. The user may use these formulations in diluted or undiluted form. Suitable injectable formulations are either powders, pellets or granules which are reconstituted in a suitable solvent before use, or are ready-to-use solutions, suspensions including nanosuspensions, or the like.

Clothianidin is preferably applied via an in-feed treatment, for example, in form of a medicated fish feed. Fish feed is typically present in the form of granules or pellets; common ingredients of said pellets or granules are, for example, fishmeal, fish oil, vegetable proteins, saccharides, such as typical mono- or disaccharides, polysaccharides, such as mannans glucans or alginates, and/or other typical excipients such as pigments, vitamins, minerals, binders and the like. A clothianidin-medicated fish feed may be prepared by incorporating a suitable amount of clothianidin or a salt thereof into the fish feed product. The clothianidin may be incorporated into the feed mixture prior to pelleting. However, it is preferred to coat the pellets or granules with clothianidin. For example, commercially available fish pellets or granules are coated with a pre-mix containing the clothianidin and one or more suitable excipients such as a starch, fumed silica (Aerosil®), microcrystalline cellulose, lactose or the like. In addition, a typical preservative may be present. The concentration of clothianidin in the pre-mix may be chosen within a broad range; for example, a clothianidin concentration of from 0.001 to 10% w/w, preferably from 0.05 to 5% w/w and in particular from 0.15 to 2.5% w/w, based in each case on the entire weight of the pre-mix, has proven as valuable. The feed pellets may be coated with the pre-mix by a dry-coating method. To this end the pre-mix is mixed with the pellets so that it is uniformly distributed onto the pellets; preferably, fish oil or vegetable oil is then added to the mixture to coat the medicated pellets. In an alternative, the pre-mix is first mixed with fish or vegetable oil, which is then mixed with the pellets to disperse it onto them, and additional fish or vegetable oil is added to the coated pellets and mixed until the pellets are thoroughly coated. According to still another embodiment, the pre-mix is first dispersed in some fish or vegetable oil, said dispersion is then sprayed onto the pellets to disperse it onto them under a vacuum coating system and mixed until the pellets are thoroughly coated.

Following the addition of the active ingredient to the fish feed, the pellets or granules comprise, for example, from 0.0005 to 5% (w/w), preferably from 0.001 to 2.5% (w/w), and in particular from 0.0025 to 1.25% (w/w) clothianidin, based on the entire weight of the fish feed.

A preferred in-feed treatment according to the invention comprises feeding clothianidin or a veterinary acceptable salt thereof to a fish population at a daily dose of 0.5 to 30 mg clothianidin per kg of fish biomass for a time period of 3 to 14 days, and wherein the overall amount of clothianidin applied during said time period is from 3 to 300 per kg of fish biomass.

According to this embodiment, the overall time period for the treatment against sea lice is, for example, from 3 to 14 days, preferably from 5 to 14 days, more preferably from 5 to 10 days and in particular for 7 days (1 week). During the overall treatment time, the feeding of the clothianidin is performed, for example, daily or every second day, and in particular daily.

The overall amount of clothianidin applied during the treatment is preferably from 5 to 175 mg per kg of fish biomass, more preferably from 5 to 140 mg per kg of fish biomass, even more preferably from 7 to 105 mg/kg fish biomass, and especially from 7 to 70 mg/kg fish biomass.

According to one preferred embodiment of the invention the clothianidin is administered daily, for a period of time of 3 to 14 days, preferably of 5 to 14 days, more preferably of 5 to 10 days and in particular of 7 days (1 week), wherein the daily dose is from 0.5 to 25 mg/kg fish biomass, preferably from 0.75 to 20 mg/kg fish biomass, more preferably from 1 to 15 mg/kg fish biomass, and in particular preferably from 1 to 10 mg/kg fish biomass. One particularly preferred treatment comprises administering clothianidin for 7 consecutive days with a daily dose of 0.75 to 20 mg clothianidin/kg fish biomass, (total amount of 5.25 to 140 mg/kg fish biomass) in particular 1 to 15 mg clothianidin/kg fish biomass (total amount 7 to 105 mg/kg fish biomass). A further particularly preferred treatment comprises administering clothianidin for 7 consecutive days with a daily dose of 0.75 to 15 mg clothianidin/kg fish biomass (total amount 5.25 to 105 mg/kg fish biomass), especially 1 to 10 mg clothianidin/kg fish biomass (total amount 7 to 70 mg/kg fish biomass), and in particular 5 to 10 mg clothianidin/kg fish biomass (total amount 35 to 70 mg/kg fish biomass).

According to a further embodiment of the invention clothianidin is administered once every second day, for a period of time preferably of 5 to 13 days, more preferably of 5 to 9 days, and in particular of 7 days. A particular treatment comprises treating the fish for a time period of 7 days, administering the feed comprising the clothianidin on days 1, 3, 5 and 7 and withholding any food the day prior to the treatment and on days 2, 4 and 6 of the treatment period. The concentration of clothianidin is adjusted to ensure that the same average dose per kg of fish biomass is administered over the entire treatment period than in a daily treatment. Concerning the overall amount of clothianidin applied during this pulsed treatment, the above given ranges including the preferences apply.

As an example, concerning a 7-day treatment with clothianidin applied on days 1, 3, 5 and 7, the clothianidin dose on each of said days is, for example, from 1.3 to 35 mg/kg fish biomass (total amount from 5.2 to 140 mg/kg fish biomass), preferably from 1.3 to 26 mg/kg fish biomass (total amount from 5.2 to 104 mg/kg fish biomass), even more preferably from 1.75 to 17.5 mg/kg fish biomass (total amount from 7 to 70 mg/kg fish biomass), and in particular from 8.75 to 17.5 mg/kg fish biomass (total amount from 35 to 70 mg/kg fish biomass).

When applying clothianidin to the fish according to the present invention, the fish will absorb the clothianidin and provide the therapeutic effect, i.e. the reduction or preferably complete elimination of sea lice, quickly.

In addition, the compound is safe both from a toxicological and environmental perspective, as its half-life in animals and in the environment is in each case short. Accordingly, the withdrawal period is short, the fish may be harvested and enter the human food chain soon following the last clothianidin treatment.

Clothianidin may in general be applied at any stage of the fish development as curative treatment in order to reduce or eliminate sea lice infestations of fish. In case of salmons, treatments take place advantageously whilst the fish are at sea.

According to one embodiment of the invention, a curative treatment with clothianidin as described above is carried out during the months with high sea water temperature and high sea lice pressure. Moreover, it is preferred to use clothianidin in cases, where a sea lice infestation of fish has to be cleared rapidly. According to a further embodiment of the invention, fish, in particular salmons, are treated with clothianidin at a late stage of the fish development; for example, the adult fish are cleared from sea lice shortly before being harvested.

A curative treatment of the fish with clothianidin also may be used in combination with a previous protective treatment with another sea lice control agent in order to efficaciously control sea lice infestations of the fish. Suitable protective sea lice controlling agents are, for example, benzoylurea compounds, in particular lufenuron or hexaflumuron, avermectins, for example emamectin benzoate, or organophosphates, for example dichlorvos. A suitable combination treatment of clothianidin and a further protective sea lice-controlling agent as mentioned above, in particular lufenuron or hexaflumuron, may be performed, for example, by treating the fish, in particular salmon, initially with said other sea lice-controlling agent, and thereafter, for example 1 week to 3 month or more, preferably 3 to 5 months or more, more preferably 6 months or more and in particular 8 to 12 months after the end of the treatment with said protective sea lice-controlling agent, performing a treatment with clothianidin as described above. According to a preferred embodiment of this combination treatment, the first treatment is an in-feed treatment with hexaflumuron or in particular lufenuron, or with another active ingredient with long lasting protection against sea lice such as emamectin benzoate, which takes place at the end of the fresh water phase of salmon evolution or at the beginning of their sea water phase.

Clothianidin is preferably not used in combination with another compound being used in the curative treatment of sea lice infestations, such a compound of formula

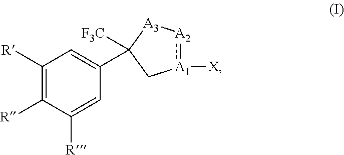

known from WO2011/157733, wherein the variables are defined as described therein.

The treatment according to the present invention may in certain cases be improved by the use of clothianidin in combination with other agents, for example a vaccine component including immune enhancing agents; or a feed ingredient containing immune modifying agents.

The following Examples further illustrate the invention.

### EXAMPLE 1

In-feed treatment of sea lice infested salmon with 5 different neonicotinoids (a) Method: Atlantic salmon (estimated mean weight 50-100 g) were batch weighed and allocated to a single 2 m diameter challenge tank. Fish were challenged with sea lice (L. salmonis) copepodids. Within one week of the successful lice challenge, fish were randomly allocated from the 2 m challenge tank to 1 m diameter treatment tanks so that each tank contained thirty fish.

Fish were maintained until lice developed to adult stages. This was monitored by periodic examination of fish from the control tank. There were five test compounds acetamiprid (A), clothianidin (B), imidacloprid (C), nitenpyram (D) plus a control (E), each compound was tested at three doses. Experimental diets were prepared by top dressing feed pellets with the powder formulation and sealing with fish oil. Once lice had developed to adult stages, fish in all tanks received an in-feed treatment according to the relevant group for a period of seven days. Uneaten feed was collected daily to estimate the mean dose achieved in each group. Fish were examined for lice ten days following the end of the treatment period.

### (b) Results

(b1) Feed Consumption

Ten grams of feed fortified with premix and fish oil were weighed and contained 133 pellets as a comparative guide for conversion to dry weight. The estimated % of nominal dose received was assessed as shown in Table 1 below. A high level of uneaten pellets may indicate a negative effect on appetite and/or low palatability of the medicated feed.

TABLE 1
Dose achieved in treatment groups based on uneaten feed

| | % of target dose | % of target | % of target |
|---|---|---|---|
| Compound | achieved | dose achieved | dose achieved |
| Nominal dose | 10 | 5 | 1 |
| [mg/kg/day] | | | |
| Control | 106 | 106 | 106 |
| Acetamiprid | 38 | 62 | 98 |
| Clothianidin | 97 | 98 | 99 |
| Imidacloprid | 33 | 77 | 100 |
| Nitenpyram | 92 | 96 | 108 |

The Table above reveals that acetamiprid and imidacloprid are unsuited for an in-feed treatment of salmon, as the food-uptake at medium and higher concentrations of added active ingredient in the food is reduced to an unacceptable level.

(b2) Efficacy Results on Salmons Infected with Sea Lice

Following the 7day treatment period, the mean sea lice count in the control group was 7±3 adult sea lice meaning the total of adult males, adult non-ovigerous females and adult ovigerous females. The overall reduction of sea lice achieved with the different active ingredients is shown in Table 2.

TABLE 2

| | Reduction of total | Reduction of total | Reduction of total |
|---|---|---|---|
| Compound | sea lice [%] | sea lice [%] | sea lice [%] |
| Dose | 10 | 5 | 1 |
| [mg/kg/day] | | | |
| Control | 0 | 0 | 0 |
| Acetamiprid | 85 | 64 | 53 |
| Clothianidin | 99 | 98 | 90 |
| Imidacloprid | 81 | 94 | 91 |
| Nitenpyram | 89 | 89 | 48 |

The Tables above reveal that clothianidin is the only compound showing an sea lice reduction efficacy of 90% or higher at all three selected concentrations. In addition, clothianidin was shown to be the only active ingredient which provided full sea lice elimination while not affecting the feed uptake.

1. Use of clothianidin or a salt thereof for reducing or eliminating sea lice in a fish population, which comprises treating the fish with clothianidin or a veterinary acceptable salt thereof at a concentration of from 3 to 300 mg clothianidin per kg of fish biomass.

2. Use according to claim 1, which comprises treating the fish population orally or by an injection, preferably by applying the clothianidin by an in-feed treatment.

3. Use according to claim 2, which comprises feeding clothianidin or a veterinary acceptable salt thereof to said fish population at a daily dose of 1 to 30 mg clothianidin per kg of fish biomass for a time period of 3 to 14 days, wherein the overall amount of clothianidin applied during said time period is from 7 to 350 mg per kg of fish biomass.

4. Use according to claim 3, wherein the clothianidin is fed to the fish population daily for 5 to 10 days, in particular for 7 days.

5. Use according to claim 2, wherein clothianidin is fed to the fish population at a daily dose of from 3 to 20 mg/kg fish biomass, in particular from 3 to 15 mg/kg fish biomass.

6. Use according to claim 1, wherein clothianidin is fed to the fish population daily for 7 days at a dose of from 3 to 15 mg/kg fish biomass.

7. Use according to claim 6, wherein the clothianidin is applied in form of a medicated fish feed.

8. Use according to claim 7, wherein the medicated fish feed is in the form of fish feed granules or pellets which are coated with clothianidin.

9. Use according to claim 2, wherein the fish population comprises salmons.

10. Use of clothianidin or a salt thereof for the preparation of a medicated fish feed for eliminating, reducing or preventing sea lice in a fish population, wherein the medicated fish feed is fed to the fish population at a daily dose of 1 to 30 mg clothianidin per kg of fish biomass for a time period of 3 to 14 days, and wherein the overall amount of clothianidin applied during said time period is from 7 to 350 mg per kg of fish biomass.

11.-15. (canceled)
